(19) United States
(12) Patent Application Publication
(10) Pub. No.: US 2012/0116044 A1
(43) Pub. Date: May 10, 2012

(54) POLYETHERESTER POLYOLS

(75) Inventors: Andreas KUNST, Ludwigshafen (DE); Berend Eling, Lemfoerde (DE); Bernd Bruchmann, Freinsheim (DE); Achim Löffler, Speyer (DE)

(73) Assignee: BASF SE, Ludwigshafen (DE)

(21) Appl. No.: 13/290,479

(22) Filed: Nov. 7, 2011

Related U.S. Application Data
(60) Provisional application No. 61/411,487, filed on Nov. 9, 2010.

(51) Int. Cl. C08G 18/48; C07C 69/708; C07C 67/24; C08G 18/08

(52) U.S. Cl. 528/77; 560/182

(57) ABSTRACT

The present invention relates to hydrophobic polyetherester polyols, to a process for preparing them, and to the use of the polyetherester polyols of the invention for producing polyurethanes.

The present invention relates to hydrophobic polyetherester polyols, to a process for preparing them, and to the use of the polyetherester polyols of the invention for producing polyurethanes.

Polyetherester polyols, in other words those polyols which have not only polyether units but also polyester units in one molecule chain, may be prepared, for example, by ring-opening polymerization of alkylene oxides on hydroxy-functional starter compounds in the presence of cyclic anhydrides. Suitable cyclic anhydrides include, for example, unsaturated anhydrides such as maleic anhydride or saturated anhydrides such as succinic anhydride, or else aromatic anhydrides such as phthalic anhydride. The copolymerization of the cyclic anhydrides with alkylene oxides such as propylene oxide takes place, as a general rule, in the presence of alkoxylation catalysts. Here, double metal cyanide catalysts in particular have been found appropriate, and result in good conversions and high incorporation rates.

Polyester polyols generally have good mechanical properties, but are sensitive to hydrolysis. Polyether polyols are generally not sensitive to hydrolysis, but usually have not very good mechanical properties.

Polyetherester polyols often combine both advantages without the attendant disadvantages. This means that, generally speaking, polyetherester polyols have good mechanical properties, without at the same time being sensitive to hydrolysis.

In numerous applications, as for example in the case of polyurethanes preparable from polyols, such as polyetherester polyols, there is a desire for hydrophobic properties. Such properties result, generally, in reduced water absorption on the part of polyurethanes prepared from the respective polyols. In polyurethanes (PU), properties of this kind are often desired. For instance, materials made from polyurethanes and having a low water absorption usually exhibit improved aging behavior in the course of service. Moreover, hydrophobically modified polyurethanes may have an altered surface structure, and this may be manifested, for example, in improved slip resistance or in a more pleasant sensation when touched (improved tactile quality). A clear advantage is offered by reduced water absorption in coatings, adhesives, sealants, and elastomers (“CASE” applications). In these applications, the maximum water absorption of the polyurethane under defined test conditions is often specified, since it is known from experience that polyurethanes with a relatively low water absorption usually exhibit improved properties in these applications. For instance, the absorption of water may reduce the hardness of the polyurethane and the attachment of the polyurethane to substrates. In casings of electronic components, there is likewise a desire for polyurethane with low water absorbency, since the absorption of water results in an increase in the dielectric constant and a reduction in the specific breakdown resistance.

These properties could be achieved, for example, through the use of hydrophobic polyols, more particularly polyetherester polyols, in a process for preparing polyurethanes.

Cited below are a number of literature examples describing the DMC-catalyzed copolymerization of cyclic anhydrides with alkylene oxides:

US2007/0265367A1 describes UV-curable polyols which are preparable by copolymerization of unsaturated acid anhydrides with propylene oxide with the assistance of DMC catalysts. Unsaturated acid anhydrides referred to in said document include cis-1,2,3,6-tetrahydrophthalic anhydride and maleic anhydride.

The document J. Appl. Polym. Sci. 2007, 103, 417 describes the copolymerization of maleic anhydride, phthalic anhydride, and succinic anhydride with propylene oxide by means of DMC catalysts.

GB 1 310 461 A1 likewise describes the copolymerization of alkylene oxides with cyclic anhydrides. Cyclic anhydrides referred to in said document included phthalic, succinic, 3,4-dichlorophthalic, tetrahydrophthalic, chlorenic, 2,3-dimethylmaleic, 4,5-dimethylphthalic, 2-phenylethylmaleic, and 2-tolylmaleic anhydrides.

It is known that it is difficult to prepare polyesterols from dicarboxylic acids and glycols which have an alkyl substituent adjacent to a reactive hydroxyl group. In the case of the preparation of polyesterols with a relatively high molecular weight, the concentration of reactive groups is low, and hence results in a reduced reaction rate. If glycols with an alkyl substituent adjacent to a hydroxyl group are used, the reaction rate toward the end of the reaction may become so low that not all of the carboxylic acid groups have undergone conversion to ester groups during the time provided for the preparation of the polyesterol. As a result, the acid number of such a polyol is generally comparatively high, higher for example than 20 mg KOH/g, and so the polyol may not be able to be used in preparing polyurethanes.

The literature available to date in the field of the preparation of polyetherester polyols, as embodied, for example, in the documents cited above, has therefore not provided any solution, or at any rate any satisfactory solution, to the problem of how to prepare polyetherester polyols having hydrophobic properties.

The object of the invention was therefore to provide hydrophobic polyols by a simple process, the intention being for the polyols preferably to have a low acid number. The products ought to deliver improved hydrophobic properties in polyurethanes, and this in turn ought to result in improved swelling values.

It has been possible to achieve this object by subjecting at least one alkylene oxide to an addition reaction with at least one H-functional starter compound with the assistance of a catalyst and in the presence of at least one alkyl-chain-substituted acid anhydride or alkyl-chain-substituted lactone, to form hydrophobic polyetherester polyols.

The present invention accordingly provides a polyetherester polyol having an acid number of less than 20 mg KOH/g, preferably less than 10 mg KOH/g, more preferably less than 5 mg KOH/g, and a composition as follows:

Y—{O—[CH₂—CHR1-O]ₘ—{[C(O)—CHR2-CHR3-X—O—]_{q}—[CH₂—CHR5-O]ₙ}_{z}—[CH₂—CHR4-O]ᵣ—H}ₛ,where
m, n and z are each integers, with m being situated in the range 0-10, n in the range 1-20, and z in the range of 1-50, and where
X is selected from ═CO or —(CH₂)ₒ—, where o is an integer and is in the range of 0-10, and where
Y is the hydrocarbon radical of a polyhydroxy-functional polyol having a functionality of 1.5-8 and an equivalent weight of 100 to 1000, preferably 100 to 500, and where
R1 is selected from the group encompassing —H; —(CH₂)ₚ—CH₃; -aryl; -cycloalkyl, where p is an integer and is in the range of 0-22, and where
R2 is selected from the group encompassing hydrogen and the aliphatic hydrocarbons having 5 to 150 carbon atoms
R3 is selected from the group encompassing hydrogen and the aliphatic hydrocarbons having 5 to 150 carbon atoms, and at least one of the two radicals R2 and R3 is not hydrogen, and where
R4 is selected from the group encompassing —H; —(CH₂)ₚ—CH₃; -aryl; -cycloalkyl, where p is an integer and is in the range of 0-22, and where
R5 is selected from the group encompassing —H; —(CH₂)ₚ—CH₃; -aryl; -cycloalkyl, where p is an integer and is in the range of 0-22, and where
q is an integer in the range from 1 to 10, r is an integer in the range from 1 to 10, and s is an integer in the range from 1 to 10.

In one preferred embodiment of the invention, X is a carbonyl unit.

In a further preferred embodiment of the invention, m is in the range 1 to 5, preferably 1 to 3.

In a further preferred embodiment of the invention, n is in the range 1 to 10.

In a further preferred embodiment of the invention, z is in the range 1 to 30.

In a further preferred embodiment of the invention, R1, R4, and R5 each independently of one another are selected from the group encompassing —H and —(CH₂)ₚ—CH₃, where p=0.

In a further preferred embodiment of the invention, at least one of the two radicals R2 and R3 is an aliphatic hydrocarbon having 16 to 22 or 50 to 70 carbon atoms.

In a further preferred embodiment of the invention, at least one of the two radicals R2 and R3 is an aliphatic hydrocarbon having 16 to 22 or 50 to 70 carbon atoms.

In a further preferred embodiment of the invention, R2 is an aliphatic hydrocarbon having 16 or 18 carbon atoms.

In a further preferred embodiment of the invention, R3 is an aliphatic hydrocarbon having 16 or 18 carbon atoms.

In a further preferred embodiment of the invention, o is in the range from 1 to 5, preferably 1 to 3, and/or q is in the range from 1 to 5.

In a further preferred embodiment of the invention, r is in the range from 1 to 5 and/or R4 is selected from the group consisting of H and —(CH₂)ₚ—CH₃, where p=0.

In a further preferred embodiment of the invention, s is in the range from 2 to 10.

In a further preferred embodiment of the invention, R1 and R5 are each —(CH₂)ₚ—CH₃, where p=0, and R4 is hydrogen.

In a further preferred embodiment of the invention, Y is the hydrocarbon radical of a polyhydroxy-functional polyol having a functionality of 1.5 to 4.

In a further preferred embodiment of the invention, X is a carbonyl unit, q is 1, n is 1 to 10, z is 1 to 10, and R1, R4, and R5 are each —(CH₂)ₚ—CH₃, and r is in the range from 1 to 5 and s in the range from 1 to 10, and one of the two radicals R2 and R3 is an aliphatic hydrocarbon having 16 or 18 carbon atoms, and the other of the two radicals R2 and R3 is hydrogen, and p=0.

In a further preferred embodiment of the invention, Y is an at least dihydroxy-functional polyol based on a natural oil.

In the present disclosure, the terms “biobased compound/biobased raw material”, “renewable compound/renewable raw material”, “natural compound/natural raw material” (such as “natural oil”, for example) are used uniformly and refer to all compounds which are not prepared from fossil raw materials, such as petroleum, natural gas or coal, in contrast to the compounds of petrochemistry, which ultimately derive from natural gas or petroleum as starting materials.

The expression “fat-based compound/fat-based raw material” refers to a specific class of biobased compounds, and describes compounds derived from fatty acids, more particularly fatty acid esters. The term “fatty acid esters” here refers to monoesters, diesters or triesters of fatty acids; the last-mentioned triesters of fatty acids are also referred to as triglycerides. Triglycerides are principal constituents of natural fats or oils, such as castor oil or soybean oil, for example.

The equivalent weight of the polyetherester polyol of the invention is preferably 400 to 6000.

The present invention further provides a process for preparing one of the polyetherester polyols of the invention defined above, by catalyzed reaction of at least one alkylene oxide with at least one H-functional starter compound in the presence of at least one alkyl-chain-substituted acid anhydride and/or alkyl-chain-substituted lactone.

The H-functional starter compounds are preferably selected from the group of the polyalcohols typically used, having a functionality F of 1.5 to 8, and their products of reaction with the abovementioned alkylene oxides. Likewise preferred, furthermore, are fat-based starter molecules such as hydroxyl-containing fats (for example, castor oil or hydroxyl-modified natural fats and oils) or hydroxy-functionalized fat derivatives (including fat-based dimer diols such as, for example, Sovermol® 908 from Cognis GmbH) The hydroxy-functionalized fat derivatives may be based, for example, on castor oil, soybean oil, palm oil or sunflower oil.

It is preferred to use exactly one H-functional starter compound.

The H-functional starter compounds stated may be prepared by means, for example, of epoxidation, ring opening, hydroformylation/hydrogenation, ozonolysis, direct oxidation or laughing-gas oxidation/reduction.

In a further preferred embodiment of the process of the invention, the H-functional starter compound is selected from the group encompassing alkylene oxide adducts of polyfunctional alcohols.

In a further preferred embodiment of the process of the invention, a DMC (double metal cyanide) catalyst is used.

DMC (double metal cyanide) catalysts used are preferably Co—Zn, Fe—Zn, and/or Ni—Zn-based double metal cyanide catalysts, particular preference being given to the use of zinc hexacyanocobaltate catalysts, as have been described in, for example, U.S. Pat. No. 3,404,109, U.S. Pat. No. 3,427,256, U.S. Pat. No. 3,427,334, U.S. Pat. No. 3,427,335, U.S. Pat. No. 3,829,505, U.S. Pat. No. 3,941,849, U.S. Pat. No. 4,472,560, U.S. Pat. No. 4,477,589, U.S. Pat. No. 5,158,922, U.S. Pat. No. 5,470,813, U.S. Pat. No. 5,482,908, U.S. Pat. No. 5,545,601, EP 0 700 949, EP 0 743 093, EP 0 761 708; WO 97/40086, WO 98/16310, WO 00/47649, and JP 4 145 123. The catalyst concentration is typically between 5 and 1000 ppm, preferably between 20 and 250 ppm, more preferably between 50 and 150 ppm, based on the total mass of the end product to be prepared.

The DMC catalyst may be introduced either directly as a solid or in suspension in a polyetherol, together with the starter compound. Suspension polyetherols used are, in general, in accordance with the prior art, alkylene oxide adducts of alcohols having a functionality of two, three or four, such as monopropylene glycol, dipropylene glycol, monoethylene glycol, diethylene glycol, 1,4-butanediol, glycerol, trimethylolpropane or pentaerythritol. These suspension polyetherols typically have a molecular weight of between 300 and 5000 g/mol, preferably between 300 and 1000 g/mol, and are obtained in general via the alkali-metal-catalyzed addition reaction of alkylene oxides.

In a further preferred embodiment of the process of the invention, in addition to the DMC catalyst, a co-catalyst compound is used which catalyzes esterification and/or transesterification reactions.

The co-catalyst is preferably selected from the group encompassing Lewis acids, organotin carboxylates, titanium compounds, metal oxides, and aryl oxides comprising aluminum, lithium, titanium, and lanthanides. With particular preference the co-catalyst is selected from the group encompassing titanium compounds having the general formula Ti(OR)₄, where R is an alkyl group having 1 to 4 C atoms. Examples of such include, but are not confined to, tetraethyl titanate, tetraisopropyl titanate, tetra-tert-butyl titanate, and mixtures thereof.

If a co-catalyst is present, the DMC catalyst is present preferably in an amount of 5 to 2000 ppm, more preferably 20 to 250 ppm, while the co-catalyst is present in an amount of 1 to 1000 ppm, based in each case on the total mass of the end product.

The stated amount of DMC catalyst and co-catalyst may be added at once at the same time at the beginning of the reaction, or alternatively may be added in succession in different phases of the reaction.

The term “alkyl-chain-substituted acid anhydride” or “alkyl-chain-substituted lactone” refers to acid anhydrides and lactones, respectively, having in each case at least one alkyl substituent that has 5 to 150, preferably 10 to 100, carbon atoms. The alkyl substituent in question may be straight-chain or branched.

In one preferred embodiment, said alkyl-chain-substituted acid anhydride used is alkyl-chain-substituted succinic anhydride.

The respective acid anhydride or lactone preferably comprises exactly one alkyl substituent.

In one embodiment of the process of the invention, alkyl-chain-substituted acid anhydrides used are preferably alkyl-chain-substituted succinic anhydrides, examples being C6-C20-substituted succinic anhydrides. Examples of products available commercially include Pentasize 8 or Pentasize 68 (C₁₈ alkenylsuccinic esteanhydride or C₁₆/C₁₈ alkenylsuccinic esteanhydride, respectively, from Trigon Chemie GmbH). Other examples of alkyl-substituted succinic anhydrides include the poly(isobutylene)-substituted succinic anhydrides (known generally by the abbreviation PIBSA). The PIBSA molecule ought preferably to have a molecular weight of 500-2000. One example of a product available commercially is Glissopal® SA from BASF SE.

Particular preference is given to using C16 or C18 succinic anhydrides or a combination of C16- and C18-substituted succinic anhydrides.

As mentioned, in one preferred embodiment of the process of the invention an alkyl-chain-substituted acid anhydride is used; in this case the alkyl-chain-substituted acid anhydride is preferably selected from the group encompassing alkylsuccinic anhydrides having 16 or 18 carbon atoms, polyisobutene-substituted succinic anhydride, and mixtures thereof.

Alkylene oxides which can be used include, for example, propylene oxide (PO), ethylene oxide (EO), 1,2-butylene oxide, 2,3-butylene oxide, 1,2-pentene oxide, 2,3-epoxypropyl neododecanoate (Cardura® E10P, Hexion Specialty Chemicals, Inc.) or styrene oxide. It is preferred to use exactly one alkylene oxide.

In one preferred embodiment of the process of the invention, the alkylene oxide is selected from the group encompassing butylene oxide, propylene oxide, and ethylene oxide; propylene oxide is used with particular preference.

The process of copolymerization is typically conducted such that the hydroxy-functional starter compound is introduced to the reactor together with the alkyl-substituted acid anhydride or lactone, such as succinic anhydride, and the DMC catalyst, and also, where used, the co-catalyst, and the catalyst is activated by addition of alkylene oxide. After activation has taken place, the alkylene oxides are metered in further continuously. In one embodiment of the invention, the alkyl-substituted acid anhydride, such as succinic anhydride, and/or the hydroxy-functional starter compound may be metered continuously into the reactor together with the alkylene oxides. The operation may also be conducted entirely continuously.

The reaction is carried out typically at temperatures between 80-200° C., preferably between 100 and 160° C.

The polyols of the invention are prepared by ring-opening polymerization of alkylene oxides and cyclic anhydrides and/or lactones. The polyols thus prepared are telechelics and have a well-defined molecular weight and functionality. The functionality is situated in the range between 2-8, preferably between 2-4, more preferably between 2-3. The use of DMC catalysts in the synthesis allows the preparation of polyols having relatively high molecular weights. The hydrophobic polyetherester polyols obtainable from the process of the invention generally possess OH numbers of between 15 and 200 mg KOH/g, preferably between 20 and 80 mg KOH/g.

The selection of the starter compounds, the alkylene oxides, and the alkyl-substituted cyclic acid anhydrides, and the weight fraction of the respective substances, allow the preparation of polyetherester hybrid polyols having different hydrophobic properties. Adjusting the functionality and the molecular weight makes it possible to fine-tune the polyol.

The incorporation of monomer into the polyetherester polyols of the invention can according to theory be alternating. This means that the molar ratio of epoxide to anhydride can be 1:1. In general, the molar amount of the epoxide will be greater than that of the acid anhydride. Thus, the polyetherester polyols of the invention have from 1 to 45 mol %, preferably between 2 and 30 mol %, of alkyl-substituted acid anhydride units, preferably succinic anhydride units, based on the monomer fraction.

Although not absolutely mandatory, it is in practise generally the case that the number of the hydrophobic alkyl-substituted cyclic acid anhydride to be incorporated will be dependent on the molecular weight of the acid anhydride. If, for example, PIBSA molecules with a molecular weight of 1000 are used, then generally 1-3 PIBSA monomers are incorporated per polyol molecule. If, for example C18 alkenylsuccinic anhydride (e.g., Pentasize 8 from Trigon GmbH) is used, the molar fraction may be higher.

The architecture of the polyols can be controlled further via the metering mode of the components and of the catalysts. Hence it is possible to include starter compounds, anhydrides, and the DMC catalyst, along with any Lewis acid catalysts, in the initial charge to the reactor, and to add the alkylene oxides continuously. Depending on the chosen amount of catalyst and the reaction temperature, the two ring-opening polymerizations may run more or less parallel alongside one another. If, however, starter compound, anhydride, and, if used, Lewis acid catalyst is added to the reactor first of all, the initial product formed from the reaction between anhydride and starter compound is the monoester. After a further addition of the DMC catalyst and of the alkylene oxides, the alkoxylation reaction that forms ether groups will also run.

The present invention further provides a process for producing polyurethane materials, wherein a) organic polyisocyanates are mixed to form a reaction mixture with b1) polyetheresterols of the invention, optionally b2) further polyols, and also chain extenders and/or crosslinking agents, c) blowing agents, d) catalysts, and optionally e) auxiliaries and additives, and this reaction mixture is reacted, the polyols (b2) being selected from the group encompassing polyetherols (b2i), polyesterols (b2ii), polycarbonate polyols (b2iii) and polyacrylate polyols (b2iv).

The present invention further provides for the use of polyetherester polyols of the invention for producing polyurethane materials.

The polyisocyanates a) used for producing the polyurethane materials of the invention comprise compounds based on methanediphenyl diiosocyanate (referred to below as MDI), toluene diisocyanate, isophorone diisocyanate, naphthalene diisocyanate, H12MDI or hexamethylene diisocyanate. MDI comprehends 2,4-MDI, 4,4′-MDI, and higher polycyclic homologs and also mixtures thereof.

The polyisocyanate a) may be used in the form of polyisocyanate prepolymers. The polyisocyanate prepolymers are obtainable by reacting above-described MDI, at temperatures, for example, of 30 to 100° C., preferably at around 80° C., with inventive polyetheresterols (b1), polyetherols (b2i) and/or polyesterols (b2ii) to form the prepolymer. Polyetheresterols (b1) used are preferably the polyetherester polyols described above. For MDI-based prepolymers, for example, the NCO content of the prepolymers is preferably in the range from 2% to 30%, more preferably from 5% to 28%, and more particularly from 10% to 25%.

Polyetherols (b2i) may be, for example, polyols having OH values of 10-800 mg KOH/g, preferably 20-100 mg KOH/g, more particularly 25-50 mg KOH/g. Alkylene oxides which can be used in preparing the polyols (b2i) may be, for example, propylene oxide (PO), ethylene oxide (EO), 1,2-butylene oxide, 2,3-butylene oxide, 1,2-pentene oxide, or styrene oxide, preference being given to propylene oxide and ethylene oxide. Particularly suitable are propylene oxide-based polyols having an ethylene oxide cap of 10 to 30% by weight, more particularly 13% to 23%, and an average functionality of 2-6, more particularly 3-5. By average functionality here is meant the average functionality of the starter compounds or of the mixture thereof. Starter compounds which can be used include, for example, glycerol, trimethylolpropane, pentaerythritol, sorbitol, sucrose, triethanolamine, and ethylenediamine or mixtures thereof. PolyTHF (polytetrahydrofuran) as well may be used as component (b2i).

Suitable polyesterpolyols (b2ii) may be prepared, for example, from organic dicarboxylic acids having 2 to 12 carbon atoms, preferably aliphatic dicarboxylic acids having 4 to 6 carbon atoms, and polyhydric alcohols, preferably diols, having 2 to 12 carbon atoms, preferably 2 to 6 carbon atoms, by conventional methods. Typically the organic polycarboxylic acids and/or derivatives thereof and polyhydric alcohols are subjected to polycondensation, advantageously in a molar ratio of 1:1 to 1:1.8, preferably of 1:1.05 to 1:1.2, without catalyst or, preferably, in the presence of esterification catalysts, usefully in an atmosphere of inert gas, such as, for example, nitrogen, carbon monoxide, helium, argon, etc., in the melt at temperatures from 150 to 250° C., preferably 180 to 220° C., optionally under reduced pressure, until the desired acid number is reached, which is advantageously less than 10, preferably less than 2.

Polycaprolactones as well may be used as component (b2ii).

As chain extenders and/or crosslinking agents it is possible to use diols and/or triols having molecular weights of less than 400 g/mol, preferably 60 to 300 g/mol. Examples of those contemplated include aliphatic, cycloaliphatic and/or araliphatic diols having 2 to 14, preferably 4 to 10, carbon atoms, such as, for example, ethylene glycol, 1,3-propanediol, 1,10-decanediol, o-, m-, and p-dihydroxycyclohexane, diethylene glycol, dipropylene glycol, and preferably 1,4-butanediol, 1,6-hexanediol, and bis(2-hydroxyethyl)hydroquinone, triols, such as 1,2,4- and 1,3,5-trihydroxycyclohexane, triethanolamine, diethanolamine, glycerol, and trimethylolpropane, and low molecular mass, hydroxyl-containing polyalkylene oxides based on ethylene oxide and/or 1,2-propylene oxide and the aforementioned diols and/or triols as starter molecules.

Where chain extenders, crosslinking agents or mixtures thereof find application in the preparation of the polyurethanes in accordance with the invention, these chain extenders and crosslinking agents are employed usefully in an amount of up to 10% by weight, based on the weight of the sum of the polyol compounds.

As blowing agents (c) it is possible to use the hydrochlorofluorocarbons (HCFCs) that are general knowledge from polyurethane chemistry, and also highly fluorinated and/or perfluorinated hydrocarbons. In accordance with the invention it is possible additionally to make use, in particular, of aliphatic and/or cycloaliphatic hydrocarbons, more particularly pentane and cyclopentane, or of acetals, such as, for example, methylal, and CO₂, as blowing agents. These physical blowing agents are typically added to the polyol component. Alternatively they may be added to the isocyanate component, or, as a combination, both to the polyol component and to the isocyanate component.

Furthermore, it is possible and customary to add water as a polyol component blowing agent, in an amount of 0.5% to 15% by weight, preferably 1% to 5% by weight, based on the total weight of the components to be used. The addition of water may be made in combination with the use of the other blowing agents described.

For the purposes of the invention it is preferred to utilize water as blowing agent.

As catalysts (d) for preparing the polyurethanes, use is made in particular of compounds which greatly accelerate the reaction of the compounds comprising reactive hydrogen atoms, more particularly hydroxyl groups, with the organic, optionally modified polyisocyanates. Compounds of this kind that are contemplated include organic metal compounds, preferably organic tin compounds, such as tin(II) salts of organic carboxylic acids, e.g., tin(II) acetate, tin(II) octoate, tin(II) ethylhexoate, and tin(II) laurate, and the dialkyltin(IV) salts of organic carboxylic acids.

Suitable examples include dibutyltin diacetate, dibutyltin dilaurate, dibutyltin maleate, and dioctyltin diacetate. The organic metal compounds are used alone or, preferably, in combination with strongly basic amines. Examples that may be mentioned include amidine, such as 2,3-dimethyl-3,4,5,6-tetrahydropyrimidine, tertiary amines, such as dimethylcyclohexylamine, triethylenediamine, triethylamine, tributylamine, dimethylbenzylamine, N-methyl-, N-ethyl-, N-cyclohexylmorpholine, N,N,N′,N′-tetramethylethylenediamine, N,N,N′,N′-tetramethylbutanediamine, N,N,N′,N′-tetramethylhexane-1,6-diamine, pentamethyldiethylenetriamine, tetramethyldiaminoethyl ether, bis(dimethylaminopropyl)urea, dimethylpiperazine, 1,2-dimethylimidazole, 1-azabicyclo[3.3.0]octane, and, preferably, 1,4-diazabicyclo[2.2.2]octane, and aminoalkanol compounds, such as triethanolamine, triisopropanolamine, N-methyl- and N-ethyl-diethanolamine, and dimethylethanolamine.

Further catalysts contemplated include the following: tris(dialkylaminoalkyl)-s-hexahydrotriazines, more particularly tris(N,N-dimethylaminopropyl)-s-hexahydrotriazine, tetraalkylammonium hydroxides, such as tetramethylammonium hydroxide, alkali metal hydroxide, such as sodium hydroxide, and alkali metal alkoxides, such as sodium methoxide and potassium isopropoxide, and also alkali metal salts of long-chain fatty acids having 10 to 20 C atoms and optionally pendant OH groups.

It is preferred to use 0.001% to 5% by weight, more particularly 0.05 to 2% by weight, of catalyst or catalyst combination, based on the weight of the synthesis components.

The reaction mixture for the inventive preparation of the polyurethanes may optionally be further admixed with other auxiliaries and/or additives (e). Examples that may be mentioned include flame retardants, stabilizers, fillers, dyes, pigments, and hydrolysis inhibitors, and also substances having a fungistatic and bacteriostatic activity.

Examples of suitable flame retardants include tricresyl phosphate, tris(2-chloroethyl)phosphate, tris(2-chloropropyl)phosphate, tetrakis(2-chloroethyl)ethylenediphosphate, dimethyl methanephosphonate, diethyl diethanolaminomethylphosphonate, and also commercial halogenated and halogen-free flame retardants. Besides the halogen-substituted phosphates already identified, use may also be made of organic or inorganic flame retardants, such as red phosphorus, aluminum oxide hydrate, antimony trioxide, arsenic oxide, ammonium polyphosphate, and calcium sulfate, expanded graphite or cyanuric acid derivatives, such as melamine, for example, or mixtures of at least two flame retardants, such as, for example, ammonium polyphosphates and melamine, and also, optionally, corn starch, or ammonium polyphosphate, melamine, and expanded graphite, and/or optionally aromatic polyester for providing the polyisocyanate polyaddition products with flame retardancy. Particularly effective in this context are found to be additions of melamine. Generally speaking, it has been found useful to use 5% to 50% by weight, preferably 5% to 30% by weight, of the stated flame retardants for 100% by weight in each case of the other components employed.

Stabilizers used are, in particular, surface-active substances, in other words compounds which act to support the homogenization of the starting materials and which, optionally, are also suitable for regulating the cell structure of the polyurethane. Examples that may be mentioned include emulsifiers, such as the sodium salts of castor oil sulfates or fatty acids, and also salts of fatty acids with amines, e.g., diethylamine oleate, diethanolamine stearate, diethanolamine ricinoleate, and salts of sulfonic acids, e.g., alkali metal salts or ammonium salts of dodecylbenzene- or dinaphthylmethane-disulfonic acid and ricinoleic acid; foam stabilizers, such as siloxane-oxalkylene copolymers and other organopolysiloxanes, oxethylated alkylphenols, oxethylated fatty alcohols, liquid paraffins, castor oil esters and ricinoleic esters, turkey red oil and peanut oil, and cell regulators, such as paraffins, fatty alcohols, and dimethylpolysiloxanes. Stabilizers employed are predominantly organopolysiloxanes which are water-soluble. These are polydimethylsiloxane radicals grafted to which there is a polyether chain comprising ethylene oxide and propylene oxide. The surface-active substances are used typically in amounts of 0.01% to 5% by weight, based on 100% by weight of the other components employed.

Fillers, more particularly reinforcing fillers, are the conventional, typical organic and inorganic fillers, reinforcing agents, weighting agents, agents for improving abrasion performance in paints, coating materials, etc. Specifically, mention may be made, by way of example, of the following: inorganic fillers, such as silicatic minerals, examples being phyllosilicates, such as antigorite, serpentine, horn blends, amphibole, chrysotile, and talc, metal oxides, such as kaolin, aluminum oxides, titanium oxides, and iron oxides, metal salts, such as chalk, heavy spar, and inorganic pigments, such as cadmium sulfide and zinc sulfide, and also glass, etc. Preference is given to using kaolin (China clay), aluminum silicate, and coprecipitates of barium sulfate and aluminum silicate, and also natural and synthetic minerals in fiber form, such as wollastonite, metal fibers and more particularly glass fibers in different lengths, these fibers possibly and optionally being sized. Examples of organic fillers contemplated are the following: charcoal, rosin, cyclopentadienyl resins, and graft polymers, and also cellulosic fibers, polyamide fibers, polyacrylonitrile fibers, polyurethane fibers, and polyester fibers that are based on aromatic and/or aliphatic dicarboxylic esters, and more particularly carbon fibers. The organic and inorganic fillers may be used individually or as mixtures and are inserted into the reaction mixture advantageously in amounts of 0.5% to 50% by weight, preferably 1% to 40% by weight, based on the weight of the other components employed, although the natural and synthetic fiber content of mats, webs, and fabrics may reach levels of up to 80% by weight.

Further details on the other customary auxiliaries and adjuvants identified above, and also on blowing agents, surfactants, and catalysts, can be found in the literature of the art, as for example in the monograph by J. H. Saunders and K. C. Frisch “High Polymers”, Volume XVI, “Polyurethanes”, parts 1 and 2, Interscience Publishers 1962 and 1964, in the above-cited Kunststoffhandbuch, “Polyurethane”, Volume VII, Hanser-Verlag Munich, Vienna, editions 1 to 3, or The Polyurethanes book, Randall and Lee, Eds, Wiley, 2002. The polyurethane materials of the invention are produced by the one-shot or prepolymer process, using low-pressure or high-pressure technology.

Where foams are concerned, the foams may be produced as slabstock foam or as molded foam. Where compact materials are concerned, a variety of casting techniques may be employed. These procedures are described in The Polyurethanes book, Randall and Lee, Eds, Wiley, 2002, for example.

The possible uses of the polyols of the invention for polyurethane components are very diverse, since there are numerous applications where enhanced water repellency is an advantage.

The polyols of the invention may be used, for example, in foamed or compact PU materials. A principle field of application for the polyols of the invention is in the sectors of coatings, adhesives, sealants, and elastomers. The field of application of elastomers is very broad—reference here is to thermoplastic polyurethanes (TPUs), microcellular elastomers, casting elastomers, RIM elastomers, spraying elastomers, elastomeric coatings, and “millable gums”. Examples of microcellular elastomers are integral foams, footwear soles, and ancillary springs for the automobile industry (Cellasto®, BASF SE). Spraying elastomers are used primarily in coating applications. The polyols of the invention may be used, furthermore, for producing flexible foams, semi-rigid foams, and carpeting foams, and also, for example, in packaging foams, rigid foams, RIM parts such as automobile bumpers and other automobile exterior parts, for example, and synthetic leather.

The polyols of the invention may be used as described above for preparing prepolymers by reaction with diisocyanates. Hence the polyols of the invention may be used, for preparing polyurethane materials, not only by direct use in the A component of the formulations, but also in the form of a prepolymer. In this connection it should be mentioned that the prepolymer fraction in the prepolymer-polyol mixture may amount to between 10% to 90%. These prepolymer-polyol mixtures are used, for example, when the polyols of the invention are employed in moisture-curing one-component systems, such as for coating, adhesive, and sealing materials, for example.

Accordingly, polyurethanes obtainable from a polyol of the invention are also provided as a further subject of the present invention.

Below, a number of examples are given for illustrating the invention. The examples are by no means restrictive on the scope of the invention, but instead should be understood merely as illustrative examples.

### EXAMPLES

Lupranol® 1200 is a difunctional polyetherol from BASF Polyurethanes GmbH, having a hydroxyl number of 250 mg KOH/g to DIN 53240.

Lupranol® 1100 is a difunctional polyetherol from BASF Polyurethanes GmbH, having a hydroxyl number of 105 mg KOH/g to DIN 53240.

Pentasize 8 is a C18 alkenylsuccinic anhydride from Trigon GmbH.

Glissopal® SA is a poly(isobutylene)succinic anhydride with molecular weight of 1000 g/mol (PIBSA 1000) from BASF SE.

Sovermol® 908 is a fat-based dimer diol from Cognis GmbH.

For synthesis example 6, castor oil was used from Alberdingk & Boley GmbH, with the designation Albodry castor oil Pharma DAB Spezial (Hydroxyl number=165 mg KOH/g to DIN 53240).

### Synthesis Example 1

634 g of Lupranol® 1200, 1345 g of Pentasize 8, and 20.5 g of a DMC catalyst suspension (5.4% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 2360 g of propylene oxide are metered in to the reaction mixture over the course of 120 minutes. When the metering is at an end, there is a subsequent reaction until the pressure is constant. The reaction mixture is freed from the residual monomer under reduced pressure. This gives 4500 g of the polyetherester polyol of the invention, in the form of a viscous liquid.

Analysis:

Hydroxyl number=39.9 mg KOH/g DIN 53240

Viscosity=1603 mPas DIN 13421

Acid number=1.01 mg KOH/g DIN 53402

Water value=0.02% DIN 51777

### Synthesis Example 2

1017 g of Lupranol® 1200, 1356 g of Pentasize 8, and 20.8 g of a DMC catalyst suspension (5.4% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 1948 g of propylene oxide are metered in to the reaction mixture over the course of 120 minutes. When the metering is at an end, there is a subsequent reaction until the pressure is constant. The reaction mixture is freed from the residual monomer under reduced pressure. This gives 4500 g of the polyetherester polyol of the invention, in the form of a viscous liquid.

Analysis:

Hydroxyl number=56.9 mg KOH/g DIN 53240

Viscosity=931 mPas DIN 13421

Acid number=0.93 mg KOH/g DIN 53402

Water value=0.02% DIN 51777

### Synthesis example 3

In a 5 L steel autoclave, 795.4 g of Lupranol® 1200 are admixed with 1183.8 g of Pentasize 8 and the reaction mixture is heated at 150° C. for 300 minutes. The resultant acid-functionalized intermediate (acid number=107.3 mg KOH/g to DIN 53402) is subsequently reacted autocatalytically to constant pressure with 659 g of propylene oxide at 150° C. 1822.1 g of the propylene oxide-capped intermediate obtained in this way (hydroxyl number=90 mg KOH/g to DIN 53240 and acid number=0.434 mg KOH/g to DIN 53402) are subsequently admixed with 17.1 g of a DMC catalyst suspension (5.4% in Lupranol® 1100). Then, at 130° C., first 200 g of propylene oxide are metered into the reaction mixture. Following activation of the catalyst, which is manifested in a drop in pressure in combination with the release of heat, the reaction temperature is raised to 160° C. and a further 1622.1 g of propylene oxide are metered in to the reaction mixture over the course of 150 minutes. When the metering is at an end, there is a subsequent reaction until the pressure is constant. The reaction mixture is freed from the residual monomer under reduced pressure. This gives 3500 g of the polyetherester polyol of the invention, in the form of a viscous liquid.

Analysis:

Hydroxyl number=44.2 mg KOH/g DIN 53240

Viscosity=1104 mPas DIN 13421

Acid number=0.306 mg KOH/g DIN 53402

Water value=0.02% DIN 51777

### Synthesis Example 4

846 g of Sovermol® 908, 1437 g of Pentasize 8, and 22.9 g of a DMC catalyst suspension (5.4% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 2527 g of propylene oxide are metered in to the reaction mixture over the course of 120 minutes. When the metering is at an end, there is a subsequent reaction until the pressure is constant. The reaction mixture is freed from the residual monomer under reduced pressure. This gives 5000 g of the polyetherester polyol of the invention, in the form of a viscous liquid.

Analysis:

Hydroxyl number=37.0 mg KOH/g DIN 53240

Viscosity=1678 mPas DIN 13421

Acid number=0.68 mg KOH/g DIN 53402

Water value=0.01% DIN 51777

### Synthesis example 5

902 g of Lupranol® 1200, 1202 g of Glissopal® SA, and 18.5 g of a DMC catalyst suspension (5.4% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 1704 g of propylene oxide are metered in to the reaction mixture over the course of 120 minutes. When the metering is at an end, there is a subsequent reaction until the pressure is constant. The reaction mixture is freed from the residual monomer under reduced pressure. This gives 4000 g of the polyetherester polyol of the invention, in the form of a viscous liquid.

Analysis:

Hydroxyl number=61.3 mg KOH/g DIN 53240

Viscosity=1876 mPas DIN 13421

Acid number=1.45 mg KOH/g DIN 53402

Water value=0.01% DIN 51777

### Synthesis Example 6

1505 g of castor oil, 777 g of Pentasize 8, and 22.7 g of a DMC catalyst suspension (5.4% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 2485 g of propylene oxide are metered in to the reaction mixture over the course of 120 minutes. When the metering is at an end, there is a subsequent reaction until the pressure is constant. The reaction mixture is freed from the residual monomer under reduced pressure. This gives 4900 g of the polyetherester polyol of the invention, in the form of a viscous liquid.

Analysis:

Hydroxyl number=47.0 mg KOH/g DIN 53240

Viscosity=1358 mPas DIN 13421

Acid number=0.04 mg KOH/g DIN 53402

Water value=0.03% DIN 51777

### Synthesis Example 7

790 g of Lupranol® 1200, 1249 g of Pentasize 8, and 20.0 g of a DMC catalyst suspension (5.47% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 2170 g of propylene oxide are metered in to the reaction mixture over the course of 120 minutes. When the metering is at an end, there is a subsequent reaction until the pressure is constant. The reaction mixture is freed from the residual monomer under reduced pressure. This gives 4400 g of a polyetherester polyol, in the form of a viscous liquid.

Analysis:

Hydroxyl number=48.9 mg KOH/g DIN 53240

Acid number=0.66 mg KOH/g DIN 53402

Water value=0.01% DIN 51777

### Synthesis Example 8

892 g of Lupranol® 1200, 1411 g of Pentasize 8, and 0.5 g of titanium(IV) tert-butoxide and also 22.8 g of a DMC catalyst suspension (5.47% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 2479 g of propylene oxide are metered in to the reaction mixture over the course of 120 minutes. When the metering is at an end, there is a subsequent reaction until the pressure is constant. The reaction mixture is freed from the residual monomer under reduced pressure. This gives 4900 g of a polyetherester polyol, in the form of a viscous liquid.

Analysis:

Hydroxyl number=51.2 mg KOH/g DIN 53240

Viscosity=873 mPas DIN 13421

Acid number=0.27 mg KOH/g DIN 53402

Water value=0.016% DIN 51777

### Synthesis Counterexample A

915.3 g of Lupranol® 1200, 605 g of phthalic anhydride, and also 31.0 g of a DMC catalyst suspension (5.47% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 3296.3 g of propylene oxide are metered in to the reaction mixture over the course of 120 minutes. When the metering is at an end, there is a subsequent reaction until the pressure is constant. The reaction mixture is freed from the residual monomer under reduced pressure. This gives 5000 g of a polyetherester polyol, in the form of a viscous liquid.

Analysis:

Hydroxyl number=46.1 mg KOH/g DIN 53240

Viscosity=1313 mPas DIN 13421

### Synthesis Counterexample B

617.8 g of Lupranol® 3300, 481.3 g of phthalic anhydride, and also 23.1 g of a DMC catalyst suspension (5.47% in Lupranol® 1100) are charged to a 5 L reactor and inertized three times with nitrogen, and the starter compounds mixture is dried at 130° C. under reduced pressure (15 mbar) for 60 minutes. Then, at 130° C., first 200 g of propylene oxide are metered into the reaction mixture. Following activation of the catalyst, which is manifested in a drop in pressure in combination with the release of heat, a further 3512.5 g of propylene oxide are metered in to the reaction mixture over the course of 120 minutes. When the metering is at an end, there is a subsequent reaction until the pressure is constant. The reaction mixture is freed from the residual monomer under reduced pressure. This gives 4800 g of a polyetherester polyol, in the form of a viscous liquid.

Analysis:

Hydroxyl number=52.7 mg KOH/g DIN 53240

Viscosity=1594 mPas DIN 13421

Acid number=0.11 mg KOH/g DIN 53402

### Synthesis Counterexample C

1740.0 g of Pentasize 8, 961.3 g of tripropylene glycol, and 0.2 g of dibutyltin dilaurate (DBTL) are weighed out at room temperature into a 4 L four-neck flask fitted with a distillation bridge. The reaction mixture is subsequently heated at 185° C. for 14 hours under 450 mbar. After the end of reaction, the reaction mixture is cooled and the reaction product is analyzed.

Analysis:

Hydroxyl number: 56 mg KOH/g DIN 53240

Acid number: 54 mg KOH/g DIN 53402

Production of Test Specimens

Materials for Producing the Test Specimens:

Byk 080 defoamer from Byk, Wesel

Thorcat 535 mercury catalyst from Thor Chemie, Speyer

1,4-butanediol chain extender from BASF, Ludwigshafen

DPG dipropylene glycol from BASF, Ludwigshafen

Dabco 33LV amine catalyst, Air Products

Zeolite paste K—Ca—Na-zeolite A in castor oil

Isocyanate 1 A mixture of Lupranat® MP102 and Lupranat® MM103, from BASF, Ludwigshafen, in a weight ratio of 1/1. Lupranat® MP102 is a prepolymer based on 4,4′-MDI and a glycol mixture, and has an NCO value of 23.0%. Lupranat® MM103 is a carbodiimide-modified 4,4′-MDI and has an NCO value of 29.5%. The mixture has an NCO value of 26.2%.

Isocyanate 2 Lupranat® MP102

The reaction components and additives are stored and processed at room temperature. The polyol component (component A; see tables) is made up and then left to stand for around 20 minutes. The amount of isocyanate added is calculated such that the index is 99.9 or 105. Component A is stirred with the isocyanate in a Speed Mixer for 60 seconds. The mixture is poured into a heated, open mold with dimensions of 15*20*0.6 cm³. The mold temperature is 70° C. The resultant sheet remains in the hot mold for half an hour, after which it is taken from the mold. The sheets are subsequently conditioned at 80° C. for four hours. The samples are stored at room temperature for a day and then split to 2 mm and tested for mechanical properties.

Swelling Test:

A section with dimensions of 4×4 cm² is cut from the 2 mm sheet, and its mass is ascertained. The sample is then placed in a 2.5 L glass vessel filled with water, the vessel spending 5 hours in a heating cabinet at 100° C. In order to prevent the sample rising, a mesh is used. When the glass vessel has been removed from the heating cabinet, the sample is removed and is dried off gently with filter paper. When the sample is cooled to room temperature, its mass is ascertained and from these figures a calculation is made of the degree of swelling, in percent ((m2-m1)/m1)×100%)). The measurement error is below 0.1%; differences in the measured values of 0.2% are significant.

### Application Examples 1-5

The inventive polyols of synthesis examples 1 to 5 all have a functionality of 2. They were processed as described above to form polyurethane elastomer sheets. The degrees of swelling (table 1) are in all cases below 1.2%.

TABLE 1

| Application ex. | | | 1 | 2 | 3 | 4 | 5 |
|---|---|---|---|---|---|---|---|
| Polyol from synthesis ex. 1 | | parts | 95 | | | | |
| Polyol from synthesis ex. 2 | | parts | | 95 | | | |
| Polyol from synthesis ex. 3 | | parts | | | 95 | | |
| Polyol from synthesis ex. 4 | | parts | | | | 95 | |
| Polyol from synthesis ex. 5 | | parts | | | | | 95 |
| Thorcat 535 | | parts | 0.7 | 0.7 | 0.7 | 0.7 | 0.7 |
| BYK-080 | | parts | 0.3 | 0.3 | 0.3 | 0.3 | 0.3 |
| 1,4-Butanediol | | parts | 4.0 | 4.0 | 4.0 | 4.0 | 4.0 |
| Isocyanate 1 | Index = 99.9 | | | | | | |
| 2 mm sheet | Standard | | | | | | |
| Density | ISO 1183-1, | g/cm\hat{ }3 | 1.06 | 1.071 | 1.064 | 1.041 | 1.063 |
| | A | | | | | | |
| Shore hardness | DIN 53505 | MPa | 51 | 49 | 50 | 54 | 43 |
| Tensile strength | DIN 53504 | MPa | 9 | 7 | 14 | 8 | 8 |
| Elongation at break | DIN 53504 | % | 400 | 330 | 550 | 290 | 650 |
| Degree of swelling | | wt. % | 0.9 | 1.0 | 1.0 | 1.2 | 1.2 |
| | | increase | | | | | |

### Application Example 6

The polyol from synthesis example 6 has a functionality of 3. It was processed as described above to form a polyurethane elastomer sheet. The degree of swelling (table 2) is not more than 1.6%.

TABLE 2

| | | Application ex. |
|---|---|---|
| | Units | 6 |
| Polyol from synthesis | parts | 100 |
| example 6 | | |
| DPG | parts | 3.0 |
| Dabco 33 LV | parts | 0.8 |
| K—Ca—Na-Zeolite A in | parts | 5.0 |
| castor oil | | |
| Isocyanate 2 | index = | |
| | 105 | |
| Shore A hardness | MPa | 44 |
| Tensile strength | MPa | 2.0 |
| Elongation at break | % | 170 |
| Density | g/cm3 | 1.064 |
| Degree of swelling | wt. % | 1.6 |
| | increase | |

### Application Examples 7-8

The inventive polyols from synthesis examples 7 and 8 have a functionality of 2. They were processed as described above to form polyurethane elastomer sheets. The degrees of swelling (table 3) shows that the polyurethane based on the polyetherester polyol prepared in the presence of Lewis acid catalyst is significantly more hydrophobic by comparison with polyurethane based on the polyetherester polyol prepared without Lewis acid catalyst.

TABLE 3

| | | Application example | |
|---|---|---|---|
| | Units | 7 | 8 |
| Polyol from synth. ex. 7 | parts | 95.0 | |
| Polyol from synth. ex. 8 | parts | | 95 |
| Thorcat 535 | parts | 0.7 | 0.7 |
| BYK-080 | parts | 0.3 | 0.3 |
| 1,4-Butanediol | parts | 4 | 4 |
| Isocyanate 1 | | Index = 99.9 | Index = 99.9 |
| Density | g/cm3 | 1.067 | 1.067 |
| Shore hardness | MPa | 49 | 45 |
| Tensile strength | MPa | 7 | 8 |
| Elongation at break | % | 370 | 500 |
| Degree of swelling | wt. % increase | 1.1 | 0.8 |

### Application Counterexamples A and B

The polyol from synthesis counterexample A has a functionality of 2, the polyol from synthesis counterexample B a functionality of 3. These polyols were processed in the corresponding systems to give polyurethane elastomer sheets. The swelling (table 4) in water of samples A and B is significantly poorer than that of samples 1-5.

TABLE 4
Application counterexamples:

| | | Application counterexample | |
|---|---|---|---|
| | Units | A | B |
| Polyol from synthesis | parts | 95 | |
| counterexample A | | | |
| Polyol from synthesis | parts | | 100 |
| counterexample B | | | |
| DPG | parts | | 3.0 |
| Dabco 33 LV | parts | | 0.8 |
| K—Ca—Na-Zeolite A in | parts | | 5.0 |
| castor oil | | | |
| Thorcat 535 | parts | 0.7 | |
| BYK-080 | parts | 0.3 | |
| 1,4-Butanediol | parts | 4.0 | |
| Isocyanate 1 | | Index = 99.9 | |
| Isocyanate 2 | | | Index = 105 |
| Degree of swelling | wt. % increase | 2.1 | 2.7 |

### Counterexample C

It was not possible to produce a bubble-free sheet from the polyol from synthesis counterexample C. The formation of bubbles is a consequence of the high acid number.

From the experimental results, therefore, it is apparent that it is possible to prepare polyether-ester polyols which, when used in the production of a polyurethane, produce a significant enhancement of the hydrophobic properties of the polyurethane.

1. A polyetherester polyol having an acid number of less than 20 mg KOH/g and a composition as follows:
Y—{O—[CH₂—CHR1-O]ₘ—{[C(O)—CHR2-CHR3-X—O—]_{q}—[CH₂—CHR5-O]ₙ}_{z}—[CH₂—CHR4-O]ᵣ—H}ₛ,where
m, n and z are each integers, with m being situated in the range 0-10, n in the range 1-20, and z in the range of 1-50, and where
X is selected from ═CO or —(CH₂)ₒ—, where o is an integer and is in the range of 0-10, and where
Y is the hydrocarbon radical of a polyhydroxy-functional polyol having a functionality of 1.5-8 and an equivalent weight of 100 to 1000, and where
R1 is selected from the group encompassing —H; —(CH₂)ₚ—CH₃; -aryl; -cycloalkyl, where p is an integer and is in the range of 0-22, and where
R2 is selected from the group encompassing hydrogen and the aliphatic hydrocarbons having 5 to 150 carbon atoms
R3 is selected from the group encompassing hydrogen and the aliphatic hydrocarbons having 5 to 150 carbon atoms, and at least one of the two radicals R2 and R3 is not hydrogen, and where
R4 is selected from the group encompassing —H; —(CH₂)ₚ—CH₃; -aryl; -cycloalkyl, where p is an integer and is in the range of 0-22, and where
R5 is selected from the group encompassing —H; —(CH₂)ₚ—CH₃; -aryl; -cycloalkyl, where p is an integer and is in the range of 0-22, and where
q is an integer in the range from 1 to 10, r is an integer in the range from 1 to 10, and s is an integer in the range from 1 to 10.

2. The polyol according to claim 1, where X is a carbonyl unit.

3. The polyol according to either of claims 1 and 2, where m is in the range 1 to 5.

4. The polyol according to any of claims 1 to 3, where n is in the range 1 to 10.

5. The polyol according to any of claims 1 to 4, where z is in the range 1 to 30.

6. The polyol according to any of the preceding claims, where R1, R4, and R5 each independently of one another are selected from the group encompassing —H and —(CH₂)ₚ—CH₃ where p=0.

7. The polyol according to any of the preceding claims, where at least one of the two radicals R2 and R3 is an aliphatic hydrocarbon having 16 to 22 or 50 to 70 carbon atoms.

8. The polyol according to any of the preceding claims, where o is in the range from 1 to 5, preferably 1 to 3, and/or q is in the range from 1 to 5.

9. The polyol according to any of the preceding claims, where r is in the range from 1 to 5 and/or R4 is selected from the group consisting of H and —(CH₂)ₚ—CH₃ where p=0.

10. The polyol according to any of the preceding claims, where Y is the hydrocarbon radical of a polyhydroxy-functional polyol having a functionality of 1.5 to 4.

11. A process for preparing a polyetherester polyol according to any of claims 1 to 10 by catalyzed reaction of at least one alkylene oxide with at least one H-functional starter compound in the presence of at least one alkyl-chain-substituted acid anhydride and/or alkyl-chain-substituted lactone.

12. The process for preparing a polyetherester polyol according to claim 11, where the H-functional starter compound is selected from the group encompassing alkylene oxide adducts of polyfunctional alcohols.

13. The process for preparing a polyetherester polyol according to claim 11 or 12, where a DMC catalyst is used.

14. The process for preparing a polyetherester polyol according to claim 13, where further to the DMC catalyst a co-catalyst compound is used which catalyzes esterification and/or transesterification reactions.

15. The process for preparing a polyetherester polyol according to any of claims 11 to 14, where an alkyl-chain-substituted acid anhydride is used.

16. The process for preparing a polyetherester polyol according to claim 15, where the alkyl-chain-substituted acid anhydride is selected from the group encompassing alkylsuccinic anhydrides having 16 or 18 carbon atoms, polyisobutene-substituted succinic anhydride, and mixtures thereof.

17. The process for preparing a polyetherester polyol according to any of claims 11 to 16, where the alkylene oxide is selected from the group encompassing butylene oxide, propylene oxide, and ethylene oxide.

18. A process for producing polyurethane materials, wherein a) organic polyisocyanates are mixed to a reaction mixture with b1) polyetherester polyols according to any of claims 1-10, optionally b2) further polyols, and also chain extenders and/or crosslinking agents, c) blowing agents, d) catalysts, and optionally e) auxiliaries and additives, and this reaction mixture is reacted, the polyols (b2) being selected from the group encompassing polyetherols (b2i), polyesterols (b2ii), polycarbonate polyols (b2iii) and polyacrylate polyols (b2iv).

19. The use of a polyetherester polyol according to any of claims 1 to 10 for producing polyurethane materials.
